(19) United States
(12) Patent Application Publication
(10) Pub. No.: US 2004/0014055 A1
(43) Pub. Date: Jan. 22, 2004

(54) Gene coding for erbin, and diagnostic and therapeutic uses thereof

(76) Inventors: Daniel Birnbaum, Marseille (FR); Jean-Paul Borg, Marseille (FR); Ben Margolis, Ann Arbor, MI (US)

Correspondence Address: LARSON & TAYLOR, PLC, 1199 NORTH FAIRFAX STREET, SUITE 900, ALEXANDRIA, VA, 22314, US

(21) Appl. No.: 10/240,926

(22) PCT Filed: Apr. 9, 2001
(86) PCT No.: PCT/FR01/01078

(30) Foreign Application Priority Data
Apr. 7, 2000 (FR) 00 04 517

(51) Int. Cl. C12Q 1/68; C07H 21/04; C12P 21/02; C12N 5/06; C07K 14/47

(52) U.S. Cl. 435/6; 435/69.1; 435/320.1; 435/325; 530/350; 536/23.5

(57) ABSTRACT

The invention concerns a novel gene, coding for a protein named Erbin (Erbb2 Interacting protein), which interacts with the intracellular part of ERBB2/HER-2 receptors, involved in particular in the development of cancers, the diagnostic and therapeutic uses of novel nucleotide sequences and identified amino acids.

The present invention relates to a novel gene which codes for a protein designated as Erbin which interacts with the receptor ERBB2/HER2.

ERBB2/HER2 is a receptor with tyrosine kinase activity belonging to the family of EGF receptors implicated in carcinomas (epithelial cancers) affecting various human organs such as the liver, the kidney and the breast for example (Ross et al, 1999; Klapper et al, 2000). This implication is directly linked to the enzymatic activity of the receptor (called tyrosine kinase) and its intracellular signalling, as numerous studies in vitro have shown. The gene erbb2 is amplified in approximately 30% of cancers of the breast and constitutes a factor of poor prognosis in terms of the survival of the patient. The fact that the receptor is overexpressed on the tumour cells and that it has an exacerbated enzymatic activity arouses a keen interest in human therapeutics. Two approaches are currently employed to target this receptor and to diminish its tumorigenic effect in man.

The first consists of blocking the excess of ERBB2/HER2 and therefore the growth of the tumours by means of a monoclonal antibody called Herceptin® or Trastuzumab® (marketed by GENENTECH) used in breast cancers. However, this medication was only effective in half of the women who were tested. The second approach is the use of inhibitors of the enzymatic activity of the receptor. No medication utilising this approach has currently been put on the market. There is therefore a need for other means capable of modulating the activity of the receptor and/or its expression in the tumours.

The authors of the present invention have managed to identify with precision a new gene which codes for a protein which fixes to the intracellular part of the receptor, constitutes a specific new adapter of ERBB2 and is implicated in its sub-cellular localisation. This new protein has been called Erbin, for “Erbb2 interacting protein”.

Annexed hereto is a list of sequences in which the sequence SEQ ID no. 1 represents the cDNA of the Erbin gene in man corresponding to the open reading frame (ORF). This ORF (nucleotides 324 to 4436 on SEQ ID no. 1) codes for a protein of 1371 amino acids of which the sequence is presented in SEQ ID no. 2.

Three regions are defined in the protein:

amino-terminal region (residues 1 to 501) containing LRR motifs,

carboxy-terminal region (residues 1279 to 1371) comprising the PDZ domain;

central region (residues 502 to 1278). This is a specific region of Erbin, not retained in other proteins, the function of which is to fix other proteins, with enzymatic activity (phosphatase, kinase . . . ) or without enzymatic activity, implicated in the functioning of Erbin or its localisation. This protein fragment or the polypeptides comprising this fragment also form part of the invention. The association may be constitutive or modulated by a post-traductional modification of Erbin (phosphorylation . . . ). With regard to the primary sequence of this region, the domains SH3 or WW of cytosolic proteins may be fixed on the residues 971 to 977, the residues 1100 to 1105, the residues 1115 to 1121. The protein fragments or the peptides or polypeptides comprising these fragments also form part of the invention.

The annexed list of sequences also comprises a fragment of cDNA of the Erbin gene in the mouse (SEQ ID no. 3) of which the coding part (nucleotides 1 to 1485 of SEQ ID no. 3) codes for an incomplete protein of 495 amino acids, represented by SEQ ID no. 4.

This mouse peptide sequence is homologous to the peptide sequence of human Erbin (more than 80% identity at the protein or nucleotide level, between the residue 914 and the residue 1371 of the human sequence SEQ ID no. 2), with the exception of the peptide between the residues 296 and 334 of SEQ ID no. 4 (corresponding to the nucleotides 889 to 1006 on SEQ ID no. 3) which is specific to the mouse sequence. These specific murine fragments also form part of the invention.

The sequence SEQ ID no. 5 comprises a partial genomic sequence of mouse Erbin. It contains the first exon of Erbin (nucleotides 2347 to 2535 on SEQ ID no. 5, which have a 94% identity with the human sequence and code for the residues 1 to 63 of mouse Erbin on SEQ ID no. 4, 100% identical to the human sequence). The isolated nucleic acid comprising a sequence comprising the nucleotides 2347 to 2535 on SEQ ID no. 5 also forms part of the invention.

The sequence SEQ ID no. 6 is a nucleotide sequence which is found inserted between the nucleotides 3956 and 3957 of human Erbin (SEQ ID no. 1), retaining its open reading frame, in a variant.

This sequence SEQ ID no. 6 codes for the peptide SEQ ID no. 7, which is inserted between the amino acids 1211 and 1212 of SEQ ID no. 2.

The sequence SEQ ID no. 8 is a probe in 5′ of the human sequence of Erbin (nucleotides 74 to 750 of SEQ ID no. 1), whilst the sequence SEQ ID no. 9 is a probe in 3′ (nucleotides 4240 to 4600 of SEQ ID no. 1).

The sequences SEQ ID no. 10 to SEQ ID no. 20 are useful triggers for amplifying the cDNA of human Erbin by PCR, according to the following table:

| | correspondence of |
|---|---|
| | nucleotides on |
| SEQ ID no. | SEQ ID no. 1 |
| 10 | 136 to 166 |
| 11 | 174 to 205 |
| 12 | 327 to 350 |
| 13 | 2788 to 2809 |
| 14 | 2812 to 2840 |
| 15 | 2892 to 2922 |
| 16 | 2993 to 3036 |
| 17 | 3199 to 3230 |
| 18 | 3339 to 3371 |
| 19 | 4458 to 4486 |
| 20 | 4490 to 4518 |

The sequence SEQ ID no. 21 represents the last nine carboxy-terminal amino acids of ERBB2.

The object of the present invention therefore is an isolated nucleic acid comprising the sequence SEQ ID no. 1, no. 3 or no. 5. It is understood that this also includes the homologous sequences defined as:

i) sequences similar to at least 70% of the sequence SEQ ID no. 1, no. 3 or no. 5; or

ii) sequences hybridising with the sequence SEQ ID no. 1, no. 3 or no. 5 or its complementary sequence under stringent hybridisation conditions, or

iii) sequences coding for the polypeptide called Erbin, as defined previously.

Preferably a homologous nucleotide sequence according to the invention is similar to at least 75% of the sequences SEQ ID no. 1, no. 3 or no. 5, more preferably to at least 85%, or at least 90%.

In a preferred manner, such a homologous nucleotide sequence hybridises specifically to complementary sequences of the sequence SEQ ID no. 1, no. 3 or no. 5 under stringent conditions. The parameters define the conditions of stringency depending upon the temperature at which 50% of the paired strands separate (Tm).

For sequences comprising more than 30 bases, Tm is defined by the relationship: Tm=81.5+0.41(%G+C)+16.6Log(concentration of cations)−0.63(%formamide)−(600/number of bases) (Sambrook et al, 1989).

For sequences of a length less than 30 bases, Tm is defined by the relationship: Tm=4(G+C)+2(A+T).

In appropriate conditions of stringency at which the aspecific sequences do not hybridise, the hybridisation temperature can preferably be from 5 to 10° C. below Tm, and the hybridisation buffers used are preferably solutions of high ionic force such as a solution 6×SSC for example.

The term “similar sequences” employed above refers to the perfect resemblance or identity between the nucleotides compared but also to the non-perfect resemblance which is qualified as similarity. This search for similarities in the nucleic sequences distinguishes for example the purines and pyrimidines.

Therefore a nucleotide sequence homologous to the ORF represented in SEQ ID no. 1, no.3 or no. 5 includes any nucleotide sequence which differs from the sequence SEQ ID) no. 1, no. 3 or no. 5 by mutation, insertion, deletion or substitution of one or several bases, or by the degeneration of the genetic code, in so far as it codes for a polypeptide having the biological activity of Erbin as defined below.

Amongst such homologous sequences are included the sequences of genes of mammals other than man, coding for Erbin, preferably of a primate, bovine, ovine or porcine, or even a rodent as well as the allelic variants.

The present invention also relates to an isolated polypeptide, called Erbin, comprising the amino acid sequence SEQ ID no. 2 or no. 4. It is understood that this also includes the homologous sequences defined as:

i) sequences similar to at least 70% of the sequence SEQ ID no. 2 or no. 4; or

ii) sequences coded by a homologous nucleic acid sequence as defined previously, that is to say a nucleic acid sequence hybridising with the sequence SEQ ID no. 2 or no. 4 or its complementary sequence under stringent hybridisation conditions.

Here too, the term “similar sequences” employed above refers to the perfect resemblance or identity between the amino acids compared but also to the non-perfect resemblance which is qualified as similarity. This search for similarities in a polypeptide sequence takes into account the conservative substitutions which are substitutions of amino acids of the same class, such as substitutions of amino acids with non-charged side chains (such as asparagine, glutamine, serine, threonine and tyrosine), amino acids with basic side chains (such as lysine, arginine and hisstidine), amino acids with acid side chains (such as aspartic acid glutamic acid); amino acids with apolar side chains (such as glycine, alanine, valine, leucine, isoleucine, proline, phenylalanine, methionine, tryptophan and cysteine).

More generally, therefore, “homologous sequence of amino acids” is understood to mean any sequence of amino acids which differs from the sequence SEQ ID no. 2 or no. 4 by substitution, deletion and/or insertion of an amino acid or of a reduced number of amino acid, particularly by substitution of natural amino acids by non-natural amino acids or amino pseudoacids at positions such that these modifications do not significantly undermine the biological activity of Erbin.

Preferably such a homologous sequence of amino acids is similar to at least 85% of the sequence SEQ ID no. 2 or no. 4, preferably at least 95%.

The homology is generally determined using sequence analysis software (for example Sequence Analysis Software Package of the Genetics Computer Group, University of Wisconsin Biotechnology Center, 1710 University Avenue, Madison, Wis. 53705). Similar amino acid sequences are aligned in order to obtain the maximum degree of homology (i.e. identity or similarity, as defined above). For this purpose it may be necessary to introduce gaps into the sequence artificially. Once the optimum alignment is achieved, the degree of homology is established by recording all the positions for which the amino acids of the two sequences compared are identical, relative to the total number of positions.

The “biological activity of Erbin” refers to its capacity for fixing on the intracellular part of the ERBB2/HER2 receptor and to its capacity to target the receptor towards the basolateral epithelium, where it can participate in the transduction of signals.

The invention also relates to variants of Erbin, such as the following two variants:

variant designated “1302”, comprising the sequence SEQ ID no. 1 with a deletion of the nucleotides 3957 to 4163, retaining the open reading frame. The protein coded by this variant has as sequence the sequence SEQ ID no. 2 deleted from residues 1212 to 1280. The isolated polypeptide comprising the sequence of amino acids 1212 to 1280 of SEQ ID no. 2 also forms part of the invention.

variant designated “1419”, comprising the sequence SEQ ID no. 1 with an insertion between the nucleotides 3956 and 3957 of this sequence of the fragment SEQ ID no. 6 which codes for the peptide SEQ ID no. 7.

All of the references to the nucleotide sequence SEQ ID no. 1 or to the sequence of amino acids SEQ ID no. 2 in the present description extend to these variants.

The invention also relates to the peptide of sequence SEQ ID no. 7 and the nucleic acid which codes for this peptide, such as the nucleic acid of sequence SEQ ID no.6.

The polypeptide of the present invention can be synthesised by all the methods well known to the person skilled in the art. The polypeptide according to the invention can for example be synthesised by the techniques of synthesis chemistry, such as the Merryfield type of synthesis which is advantageous for reasons of purity, antigenic specificity, absence of unwanted secondary products and its ease of production.

A recombinant protein can also be produced by a process in which a vector containing a nucleic acid comprising the sequence SEQ ID no. 1 or no. 3 or a homologous sequence is transferred into a host cell which is cultured in conditions permitting the expression of the corresponding polypeptide.

The protein produced can then be recovered and purified.

The purification processes used are known to the person skilled in the art. The recombinant polypeptide obtained can be purified from lysates and cellular extracts, from the supernatant of the culture medium, by methods used individually or in combination, such as fractionation, methods of chromatography, techniques of immunoaffinity with the aid of specific monoclonal or polyclonal antibodies, etc.

The nucleic acid sequence of interest here which codes for Erbin can be inserted into an expression vector in which it is bonded in an operating manner to elements permitting the regulation of its expression, such as in particular promoters, activators and/or terminators of transcription.

The signals controlling the expression of the nucleotide sequences (promoters, activators, termination sequences . . . ) are chosen as a function of the cellular host used. For this purpose the nucleotide sequences according to the invention can be inserted into vectors with autonomous replication within the chosen host or vectors integrating with the chosen host. Such vectors will be prepared according to the methods currently used by the person skilled in the art, and the clones resulting therefrom can be introduced into an appropriate host by standard methods, such as for example electroporation or precipitation with calcium phosphate.

The cloning and/or expression vectors such as are described above, containing a nucleotide sequence defined according to the invention, also form part of the present invention.

The invention also relates to host cells transfected, in a transitory or stable manner, by these expression vectors. These cells can be obtained by the introduction into the host cells, prokaryotes or eukaryotes, of a nucleotide sequence inserted into a vector such as is defined above, then the culturing of the said cells in conditions permitting the replication and/or the expression of the transfected nucleotide sequence.

Examples of host cells include in particular cells of mammals, such as the cells COS-7, 293, MDCK, cells of insects such as the cells SF9, of bacteria such as E. coli and of strains of yeasts such as L40 and Y90.

The nucleotide sequences of the invention may or may not be of artificial origin. They may be DNA or RNA sequences, obtained by screening of banks of sequences by means of probes developed on the basis of the sequence SEQ ID no. 1, no. 3, no. 5 or 6. Such banks can be prepared by conventional techniques of molecular biology which are known to the person skilled in the art.

The nucleotide sequences according to the invention can also be prepared by chemical synthesis, or even by mixed methods including the chemical or enzymatic modification of sequences obtained by screening of the banks.

The nucleotide sequence of the invention permits the production of probes or primers which hybridise specifically with the sequence SEQ ID no. 1, no. 3, no. 5 or 6 according to the invention, or its complementary strand. The appropriate hybridisation conditions correspond to the conditions of temperature and of ionic force usually used by the person skilled in the art, preferably under stringent conditions such as have been defined previously. These probes can be used as a diagnostic tool in vitro for the detection, by hybridisation experiments, notably by hybridisation “in situ”, of specific transcripts of the polypeptide of the invention in biological specimens or for demonstrating aberrant syntheses or genetic anomalies resulting from a polymorphism, mutations or poor splicing.

The nucleic acids of the invention which are useful as probes have a minimum of 15 nucleotides, preferably at least 20 nucleotides, more preferably at least 100 nucleotides.

The nucleic acids which are useful as primers have a minimum of 15 nucleotides, preferably at least 18 nucleotides, more preferably at least 40 nucleotides.

More precisely, the present invention relates to a nucleic acid having at least 15 nucleotides, which hybridises specifically with one of the nucleic acid sequences SEQ ID no. 1, or its complementary one, in stringent hybridisation conditions.

The nucleic acids consisting of the sequences SEQ ID no. 8 or SEQ ID no. SEQ ID no.9 can be used for example as probe.

It is also possible to use as probe the fragments of nucleic acid from the nucleotides 1 to 750 of SEQ ID no. 1 and the fragments of nucleic acid from the nucleotides 4240 to 6412 of SEQ ID no. 1.

It is also possible to use as a primer, for amplification (for example by PCR), the nucleic acids consisting of the sequences SEQ ID no. 10 to SEQ ID no. 20.

The probes or primers according to the invention are preferably labeled prior to their use. For this, various techniques are within the scope of the person skilled in the art, such as for example fluorescent, radioactive, chemicoluminescent or enzymatic labeling.

The methods of diagnosis in vitro in which these oligonucleotides are employed for the detection of mutations or of genomic modifications, at the level of the Erbin gene, are included in the present invention.

The person skilled in the art knows well the standard methods for analysing the DNA contained in a biological specimen and for diagnosing a genetic disorder. Numerous strategies for genotype analysis are available (Antonarakis et al, 1989; Cooper et al, 1991).

The DGGE method (denaturing gradient gel electrophoresis), the SSCP method (single strand conformation polymorphism) or the DHPLC method (denaturing high performance liquid chromatography; Kuklin et al, 1997; Huber et al, 1995) may preferably be used for detecting an anomaly in the Erbin gene. Such methods are preferably followed by direct sequencing. The RT-PCR process can be advantageously employed for detecting anomalies in the transcript of the Erbin, since it makes it possible to display the consequences of a splicing mutation causing the loss of one or several exons at the level of the transcript, or an aberrant splicing due to the activation of a cryptic site. This process is also preferably followed by direct sequencing. The most recently developed processes using DNA chips can also be employed for detecting an anomaly in the Erbin gene (Bellis et al, 1997).

The cloning of the Erbin gene as well as the identification of various mutations responsible for the development of tumours make it possible to envisage direct diagnosis. The present invention therefore relates to the use of at least one nucleic acid such as has been defined previously for the detection of an anomaly in the Erbin gene, defined as comprising a nucleic acid sequence SEQ ID no. 1, no. 3, no. 5 or no. 6 in its transcript.

Consequently the invention relates to a process for diagnosing in vitro a tumour or a predisposition to develop a tumour, comprising the steps consisting of:

putting a biological specimen containing DNA or RNA into the presence of specific oligonucleotides permitting amplification of all or part of the Erbin gene or of its transcript, defined as comprising a sequence SEQ ID no. 1, no. 3, no. 5 or no. 6;

amplifying said DNA or RNA;

detecting the products of amplification;

comparing the products of amplification which are obtained with those obtained with a control specimen, and in this way detecting a possible anomaly in the Erbin gene or in its transcript, indicating a predisposition to develop a tumour.

The biological specimen in question can in particular be blood, or a fragment of tissue taken from a patient.

Since the expression of Erbin is found on cancerous cells, the methods of evaluating the expression of this protein from suspect tissue samples taken from patients are also particularly advantageous in diagnostic tests in anatomopathology.

These methods can aim to detect the mRNA coding for Erbin or the protein Erbin itself.

According to the first embodiment, the invention relates to a method of diagnosing in vitro a tumour or a predisposition to develop a tumour, comprising the steps consisting of:

putting a biological specimen containing mRNA, obtained by taking a sample of suspect cells from a patient, into the presence of specific oligonucleotides permitting amplification of all or part of the Erbin gene or of its transcript, defined as comprising a sequence SEQ ID no. 1, no. 3, no. 5 or no. 6;

amplifying said transcript;

detecting and quantifying the products of amplification;

a modification in the amount of Erbin transcript with reference to the normal control indicating a tumour or a predisposition to develop a tumour.

According to a second embodiment, the invention relates to an in vitro method for the detection or measurement of the rate of expression of Erbin in a biological sample, comprising contacting at least one antibody directed against Erbin with said biological sample in conditions permitting the possible formation of specific immunological complexes between Erbin and said antibody or antibodies and the detection of the specific immunological complexes which may be formed.

The invention relates more particularly to the implementation of the above method for the in vitro diagnosis of a tumour or of a predisposition to develop a tumour on a biological specimen obtained from a sample of suspect cells taken from a patient.

The presence of the Erbin protein or of the transcript of the gene in quantities which are different from normal may be correlated with a more or less serious prognosis, in terms of the aggressiveness of the tumour for example.

The invention also relates to antibodies directed against the polypeptide Erbin such as defined previously, or against the specific fragments thereof, such as the fragment defined between the residues 502 to 1278 of the sequence SEQ ID no. 2, or advantageously against the fragment defined between the residues 914 and 1371 of the sequence SEQ ID no. 2.

These may be polyclonal or monoclonal antibodies or fragments thereof, chimeric antibodies, particularly humanised or immunoconjugated ones.

The polyclonal antibodies can be obtained from the serum of an animal immunised against a polypeptide according to the usual operating methods.

According to one embodiment of the invention, it is possible to use as antigen an appropriate peptide fragment, such as has been defined above, capable of being coupled through the intermediary of a reactive residue to a protein or another peptide. Rabbits are immunised with the equivalent of 1 mg of the peptide antigen according to the process described by Benoit et al (1982). At four-weekly intervals the animals are treated by injections of 200 μg of antigen and bled 10 to 14 days later. After the third injection, the antiserum is examined to determine its capacity to bond to the antigenic peptide radio-marked with iodine, prepared by the chloramine-T method and is then purified by carboxymethyl cellulose (CMC) ion exchange column chromatography. The antibody molecules are then collected in the mammals and isolated until the desired concentration is reached using methods well known to the person skilled in the art, for example by using DEAE Sephadex to obtain the IgG fraction.

In order to increase the specificity of the polyclonal serum, the antibodies can be purified by immunoaffinity chromatography using immunising polypeptides on solid phase. The antibody is contacted with the immunising polypeptide on solid phase for a sufficient duration so as to cause the polypeptide to immunoreact with the antibody molecule in order to form an immunological complex in the solid phase.

The monoclonal antibodies can be obtained according to the conventional method of hybridome culture described by Köhler and Milstein (1975).

The antibodies or antibody fragments according to the invention can be for example chimeric antibodies, humanised antibodies, fragments Fab and F(ab′)2. They may also be presented in the form of immunoconjugates or of labeled antibodies.

The antibodies according to the invention, in particular the monoclonal antibodies, can in particular be used for the analysis by immunohistochemistry of sections of specific tissues, for example by immunofluorescence, gold labeling, immunoperoxidase . . . .

The antibodies thus produced can be advantageously employed in any situation where the expression of Erbin must be observed.

The invention relates more generally to the use of at least one antibody thus produced for the detection or the purification of a polypeptide such as has been defined previously in a biological specimen.

The invention also relates to a kit for carrying out this method, comprising:

at least one antibody specific for Erbin, possibly fixed on a support;

means for revealing the formation of specific antigen/antibody complexes between Erbin and said antibody and/or means for quantification of these complexes.

The invention also relates to a method of obtaining a transgenic non-human animal, preferably a mouse, in which the Erbin gene is invalidated. The animal which can be obtained by this process also forms part of the invention. Such an animal, designated as a “knock-out” for this gene, is a useful model for studying the susceptibility to develop tumours candidates to suppress tumours, or on the other hand to evaluate the cytotoxicity of certain molecules.

The polypeptide according to the invention or the nucleic acid coding for this polypeptide are useful by way of a medicament, particularly for the treatment of tumours in which for example the expression of a modified Erbin is observed, and for the treatment of those in which it is sought to restore the activity of the wild-type Erbin. These may be any type of tumour, including carcinomas but also the tumours affecting the brain.

The invention also relates to a pharmaceutical composition comprising a polypeptide such as has been defined previously or a nucleic acid coding for said polypeptide, in association with a pharmaceutically acceptable vehicle.

The modes of administration, the posologies and the galenic forms of the pharmaceutical compositions according to the invention, containing at least one polypeptide, can be determined in the usual manner by the person skilled in the art, particularly according to the criteria generally taken into account for the establishment of a therapeutic treatment adapted to a patient, such as for example the age or the body weight of the patient, the gravity of the patient's general condition, the tolerance of the treatment and the secondary effects observed, etc.

As a general rule, a therapeutically or prophylatically effective quantity varying from approximately 0.1 μg to approximately 1 mg can be administered to human adults.

The invention also relates to a pharmaceutical composition comprising a nucleic acid such as has been defined previously which codes for Erbin and a pharmaceutically acceptable vehicle, said composition being intended to be used in gene therapy. The nucleic acid preferably inserted into a vector, generally a viral vector (such as adenoviruses or retroviruses), can be administered in naked form, free of any vehicle favouring the transfer to the target cell, such as anionic liposomes, cationic lipids, microparticles, for example gold microparticles, precipitating agents, for example calcium phosphate, or any other agent facilitating the transfection. In this case the polynucleotide can be simply diluted in a physiologically acceptable solution, such as a sterile solution or a sterile buffer solution, in the presence of or in the absence of a vehicle.

Alternatively, a nucleic acid according to the invention can be associated with agents which facilitate the transfection. It may be, inter alia, (i) associated with a chemical agent which modifies the cell permeability such as bupivacaine; (ii) encapsulated in liposomes, possibly in the presence of supplementary substances facilitating the transfection; or (iii) associated with cationic lipids or microparticles of silica, or gold or of tungsten.

When the nucleic acid structures of the invention cover microparticles, these latter can be injected by the intradermal or intraepidermal route using the “gene gun” technique (WO 94/24263).

The quantity to be used as a medicament depends particularly upon the nucleic acid structure itself, the individual to whom this nucleic acid is administered, the mode of administration and the type of formulation, and the pathology. As a general rule, a therapeutically of prophylactically effective quantity varying from approximately 0.1 μg to approximately 1 mg, preferably from approximately 1 μg to approximately 800 μg, more preferably from approximately 25 μg to approximately 250 μg, can be administered to human adults.

The nucleic acid structures of the invention can be administered by any conventional administration route such as in particular parenterally. The choice of administration route depends in particular upon the formulation chosen. Administration targeted at the site of the tumours concerned can be particularly advantageous.

Finally, the invention relates to a therapeutic treatment method in which an effective quantity of an Erbin polypeptide such as has been defined previously or a nucleic acid coding for this polypeptide is administered within the framework of gene therapy to a patient who needs such a treatment.

The patient concerned is generally a human being, but the application can also be extended to any mammal if appropriate.

Another aspect of the invention relates to cancerous tumours in which the ERBB2/HER2 receptor is implicated, an overexpression of this receptor often being observed in these cases. Amongst these tumours may be cited more particularly the carcinomas (epithelial cancers) which affect for example the liver, the kidney, the breast or the colon.

One strategy can then be to inhibit the interaction between Erbin and the receptor in order to delocalise it.

Another strategy consists of blocking the expression of the native Erbin, for example by means of antisense oligonucleotides preventing the transcription of the naked RNA of the Erbin gene.

Therefore the invention also relates to a method of screening such molecules inhibiting the binding of Erbin and ERBB2 receptor, this method comprising contacting a molecule to be tested with

(i) a first binding partner which is the ERBB2/HER2 receptor or a fragment thereof which fixes to Erbin, and

(ii) a second binding partner which is Erbin or a fragment thereof which fixes to the receptor,

said first and/or second binding partner(s) being labeled in such a way as to be detected,

and evaluating the inhibition of the interaction between the said binding partners by the molecule to be tested.

In order to carry out these competitive binding tests, for example of the ELISA or IRMA types, a fragment of the ERBB2/HER2 receptor which fixes to Erbin can therefore be used. This fragment comprises at least the nine last C-terminal amino acids of the receptor, namely the sequence EYLGLDVPV represented by SEQ ID no. 21. On the other hand, it is possible to use recombinant Erbin or a fragment thereof, which fixes to the receptor, namely a fragment which comprises the PDZ domain of Erbin.

One or the other of these binding partners is labeled in a detectable manner, the means for labeling the proteins being well known to the person skilled in the art. This could be a fluorescent labeling agent which binds chemically to the proteins without denaturation in order to form a staining fluorochrome which is a useful immunofluorescent indicator. The labeling agent can also be an enzyme such as peroxidase (HRP) or glucose oxidase. Radioactive elements can also be used as labeling agents.

In a preferred manner a protein is used (preferably Erbin) which is fused or coupled to a protein of the type of biotin, glutation-S-transferase, polyhistidine . . . .

One or the other of the binding partners (preferably the receptor) can be immobilised in an advantageous manner on a solid phase (membrane, beads, plastic . . . ).

The interaction between the binding partners is therefore demonstrated by an enzymatic test, fluorescence, or by autoradiography, . . . .

The molecules having inhibiting activity which are selected by this method can then be subjected to a test of precipitation of the ERBB2/HER2 receptor produced in the cells of mammals. The receptor is precipitated from a cellular lysate by virtue of the recombinant protein comprising the PDZ domain of Erbin and revealed by Western Blot or by another sufficiently sensitive method.

The molecules in which the inhibiting activity of the Erbin-receptor interaction is confirmed are retained to be tested on polarised epithelial cells in order to evaluate their capacity to induce delocalisation of the receptor.

Alternatively, a triple-hybrid test in yeast can be carried out for this purpose (Zhang J. et al, 1996).

The molecules thus screened represent excellent candidates in a strategy of anti-cancer therapy consisting of inhibiting the interaction between Erbin and ERBB2 in the tumour cells in order to delocalise the receptor and to render it inactive.

Conversely, in the case where an individual expresses an altered Erbin protein or a mutated ERBB2 receptor such that the Erbin-receptor interaction is diminished relative to a normal control, it may be advantageous to seek activators of this interaction.

To this end a method is proposed for screening of molecules activating the binding of Erbin and ERBB2 receptor which can be carried out easily on the model of the method for screening of inhibitors described above or by the triple-hybrid technique.

The invention also relates to a process for identification of mutants of Erbin which increase or reduce the interaction between this protein and ERBB2, in which a mutation is effected on the Erbin and the effect of this mutation on the interaction of the latter with the ERBB2 receptor is evaluated.

The mutagenesis can be targeted or achieved by chance according to techniques known to the person skilled in the art. The evaluation of the effect of the mutation can be achieved by a test of precipitation of ERBB2 or a double-hybrid test, or even by means of an expression bank.

In this latter technique, the mutated PDZ domain is expressed by means of phages or bacteria spread over culture boxes. After transfer of the proteins produced onto a membrane of the nitrocellulose type, the peptide of ERBB2 labeled by radioactivity, biotinylation or some other method is incubated with the membrane. The mutants which abolish the interaction and no longer fix the peptide are taken and the nucleotide sequence is analysed in order to know the mutation introduced.

The invention also relates to the mutants of Erbin which increase or decrease the interaction between the PDZ domain of this protein and ERBB2.

The mutation of histidine (position 1347) into leucine (mutant HL) or the mutation of the residues leucine (position 1291) into methionine and phenylalanine (position 1293) into isoleucine (mutant MI) cause in particular an increase in the interaction between the PDZ domain of Erbin and ERBB2.

The mutation of histidine (position 1347) into tyrosine and of glycine (position 1348) into aspartic acid (mutant YD) or the mutation of threonine (position 1316) into asparagine and of arginine (position 1317) into serine (mutant NS) on the other hand cause a decrease in the interaction between the PDZ domain of Erbin and ERBB2.

The invention also relates to a process for identification of mutants of the ERBB2 receptor which inhibit or which increase the interaction with Erbin, in which a mutation of the ERBB2 receptor is effected and the effect of this mutation on the interaction of this receptor with Erbin is evaluated. The sequence of the human ERBB2 receptor is accessible for example on the EMBL database (access number X03363).

A double-hybrid test in yeast can be particularly appropriate for this purpose.

Thus mutations have been introduced in the last 9 carboxy-terminal amino acids of ERBB2 (peptide EYLGLDVPV) (SEQ ID no. 21). The changing of one of the amino acids underlined in the peptide, for example, into alanine, inhibits the interaction with the PDZ domain of Erbin.

The mutants of ERBB2 capable of being identified by this process also form part of the invention.

The authors of the present invention have more particularly determined that the mutants of ERBB2 mutated on the positions 5(L) and 6 (D) of SEQ ID no. 21 are incapable of binding Erbin but remain capable of binding other PDZ domains of other proteins, such as PICK1.

These mutants are in particular useful for discriminating molecules which inhibit the interaction of the PDZ domain of Erbin in screening processes.

These mutants of Erbin or of the ERBB2 receptor can be used in tests for screening other compounds which inhibit or activate the interaction between Erbin and the ERBB2 receptor. They can also be exploited within the framework of a therapy by administration of these mutated forms or of nucleic acids coding for these mutated forms.

In particular, the forms which activate the Erbin-ERBB2 interaction can be exploited as a medication which is useful in an individual who expresses an altered Erbin protein or a mutated ERBB2 receptor and in whom the Erbin-receptor interaction is reduced relative to a normal control.

Conversely, the forms which inhibit the Erbin-ERBB2 interaction can be useful as medication in the case for example of an overexpression of the receptor.

### FIGURES

FIG. 1 displays a Western Blot showing the specific interaction of the PDZ domain of Erbin with ERBB2 but not with EGF-R. The bonded proteins transferred onto nitrocellulose have been revealed by anti-ERBB2 and anti-EGF-R antibodies. One tenth of the lysate was loaded on gel (total lysate TL). The GST alone or the GST-X11α PDZ domain (X11) were used as control. The GST-Erbin polypeptides used are: GST-Erbin (914-1371) lane 1, GST-Erbin (914-1240) lane 2 and PDZ domain of GST-Erbin lane 3.

FIG. 2 shows a transfer onto a nitrocellulose membrane of a SDS-PAGE gel in which has been loaded a fusion protein formed by the last 9 amino acids of the receptors related to EGF-R fused to the GST protein. The PDZ domain of the soluble GST-Erbin labeled at ³²P or the fusion proteins GST-LIN-7 were used in order to probe the membrane. After incubation, the membranes were washed and the binded proteins were revealed by autoradiography.

FIG. 3 shows a Western Blot of total lysates of different murine tissues and cell lines subjected to electrophoresis on SDS-PAGE, transfer onto nitrocellulose and contacted with a purified anti-Erbin antibody. The Erbin is a 180 kD protein indicated by the arrows.

### EXAMPLES

### Example 1

Cloning of Erbin, a Gene Coding for a Protein with PDZ Domain Interacting with the ERBB2 Receptor.

The ERBB receptors receive their ligands, coming from the stroma, on the basolateral epithelium where they are localised (Klapper et al, 2000). The analysis of the carboxy-terminal peptide sequence of the ERBB receptors has revealed that the ERBB2 receptor contained a site for bonding to a PDZ domain preserved among humans, mice, quails and dogs (Songyang et al, 1997).

The authors of the invention then screened a library of mouse kidney cDNA using the two-hybrids system with the last 9 amino acids of ERBB2 fused with the binding domain to the GAL4 DNA (“GAL-BD”).

Two-Hybrids Procedure

The last nine amino acids of ERBB2 were fused to the sub-unit GAL4-BD using the vector pc97 which carries LEU2. A bank of embryonal mouse kidney cDNA primed with an oligo-dT sequence, cloned in a vector pc86 which carries the marker Trp1 as selection marker was screened using the bait GAL4-BD-ERBB2 and the strain of yeast Y190 according to the protocol with lithium-acetate. Approximately 10⁶ transformants TRP*LEU* were selected on agar plates containing a medium deficient in tryptophan, leucine and histidine during primary screening and contain 10 mM of 3-aminotriazole (3AT). The β-galactosidase activity was tested by transferring the yeasts onto filters during the secondary screening.

After recovery, the DNA of the selected clones was retransformed in a yeast Y190 containing GAL4-BD-ERBB2 or GAL4-BD fused to control peptides.

In an alternative manner, the last 15 amino acids of ERBB2, of mutated ERBB2 (mutant VA: mutant in which the valine residue of the C-terminal end of ERBB2, a crucial residue for the interaction at the PDZ domain, has been mutated to alanine) and ERBB4 were fused to the protein LexA using the vector pBTM116 which carries Trp1. The PDZ domain of Erbin was fused to the region GAL4-AD coded by the vector pACT2 which carries Leu. Interactions were achieved in the strain of yeast of L40.

Procedures Relating to the Proteins

The cells were washed twice with a cold phosphate buffer solution PBS and lysed in a lysis buffer (50 mM HEPES pH 7.5, 10% glycerol, 150 mM NaCl, 1% triton X-100, 1.5 mM MgCl₂, 1 mM EGTA) complemented with 1 mM of PMSF (phenylmethylsulphonyl fluoride), 10 μg/ml of aprotinine and 10 μg/ml of leupeptine. Sodium orthovanadate with 200 μM final concentration was added to the lysis buffer when the cells had been stimulated with EGF. After centrifugation at 16000 g for 20 minutes, the protein content of the lysate was standardised utilising the protein dosage kit from Bio-Rad. For the immunoprecipitation, the lysates were incubated with antibodies for one night at 4° C. A-agarose protein was added and the immune complexes binded to the beads were recovered after one hour, washed three times in HNTG buffer (50 mM HEPES pH 7.5, 10% glycerol, 150 mM NaCl, 0.1% Triton X-100), boiled in test buffer IX and separated by SDS-PAGE. The transfer and the imunoblot on nitrocellulose using the chemicoluminescence method with the aid of the anti-rabbit HRP antibodies or anti-mouse HRP antibodies were carried out as described by Borg et al, 1996. For the tests known as “Far Western”, the membrane was incubated for two hours at ambient temperature with soluble GST fusion proteins marked with the protein kinase A and ³²PγATP diluted in skimmed milk TBS at 5%, 1 mM DTT (10⁶ cpm/ml). After rinsing in TBS-0.1% Triton X-100 buffer and TBS buffer, the bonded GST was revealed by autoradiography. The cellular transfection, the production of GST and the bonding dosages of GST were carried out as described previously (Borg et al, 1996).

The fractionated lysates were prepared as follows: the cells were lysed in hypotonic buffer (10 mM Tris-Cl [pH 7.4], 0.2 mM MgCl₂, 5 mM KCl) complemented with 1 mM of PMSF, 10 μg/ml of aprotonine and 10 μg/ml of leupeptine. The lysis was completed by 20 passages in a cold homogeniser (piston B). Saccharose was added to the homogenate, called the cytoplasmic fraction. The deposit was resuspended in a volume of lysis buffer (such as has been described above) equivalent to the volume of cytoplasmic fraction. After 30 minutes on ice, the insoluble debris was removed by centrifugation at 16000 g for 30 minutes at 4° C. The resulting supernatant was called the membranal fraction.

Results:

Thus the authors of the invention isolate a partial cDNA clone coding for the last 457 amino acids of a novel protein called Erbin for “ERBB2 interacting protein”. The clone drawn from the kidney bank of GAL-4-BD-ERBB2 contains the residues 914 to 1371 of murine Erbin and is called Erbin (914-1371). The proteins of fusion with GST are produced using the peptide sequences of Erbin (914-1371), Erbin (914-1240) and Erbin (1240-1371). The protein of fusion GST-Erbin (1240-1371) will be designated in the text as the PDZ domain of GST-Erbin.

The strain of yeast Y190 contransformed by vectors coding for the bait fused to Lex A and the prey fused to GAL4-BD were seeded on Trp-Leu-His medium containing 10 mM of 3AT. In the following table the + signs signify growth on the selective medium (-His) and positive β-galactosidase activity.

| pACT-Erbin | 914-1371 | | 1240-1371 | |
|---|---|---|---|---|
| LexA | βgal | −His | Bgal | −His |
| ERBB2 | + | + | + | + |
| ERBB2.VA | − | − | − | − |
| ERBB4 | − | − | − | − |

No interaction is observed with mutated ERBB2 on the C-terminal valine residue. The PDZ domain found at the C-terminal end of Erbin is sufficient to bond to ERBB2.

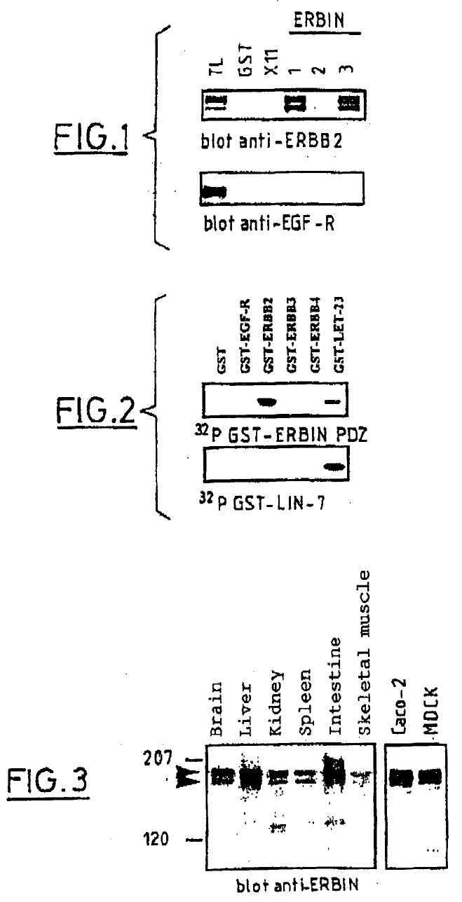

No bonding was observed with EPHB2, MUSK, PDGFRα and PDGFRβ. The proteins GST-Erbin (914-1371) and GST-Erbin PDZ bond to the chimera EGF-R/ERBB2 (HER 1/2) but not to EGF-R in “pull-down” tests of precipitation of proteins (FIG. 1). The domains GST-DLG, LIN-2 and X11α do not bond to HER 1/2. The direct binding of the PDZ domain of Erbin to the carboxy-terminal ends of the receptors ERBB was evaluated by “Far Western” test. The PDZ domain of the radiomarked Erbin bonds to the peptide ERBB2 but not to the peptide EGF-R, ERBB3 and ERBB4 and only bonds weakly to the peptide LET-23 (FIG. 2). The PDZ domain of LIN-7 bonds solely to LET-23. The PDZ domain of Erbin has a strong identity with the PDZ domain of DENSIN-180 (71%) and a 35% identity with the PDZ domains of class I (Songyang et al, 1997; Fanning et al, 1998) found in the proteins LIN-7 and PSD-95 according to the alignment of the sequences. The substitution of a His-Gly motif present in the αB motif of Erbin into a Tyr-Asp motif present in the PDZ domain of class III of NOS inhibits the interaction with ERBB2. These results show that the PDZ domain of Erbin interacts specifically and directly with ERBB2.

A cDNA of full length coding for human Erbin was cloned by RT-PCR from Daudi, a cellular line of human B lymphocytes, using the MARATHON kit, according to the recommendations of the manufacturer (Clontech). An open reading frame by stop codons in the three frames codes for a protein of 1371 amino acids. Erbin contains 16 LRR motifs in its amino-terminal end (residues 48-416) and one single C-terminal PDZ domain. A wide intermediate region of 863 amino acids between the LRR and PDZ domains contains extensions rich in proline which can represent binding sites or SH3 and WW domains.

### Example 2

Characterisation of Erbin, Member of a New PDZ Family

A specific human Erbin probe has permitted detection of a transcript of 7.2 kb in the majority of human and murine tissues tested. This analysis was carried out by Northern Blot with the aid of the “Multiple Tissues” technique of Clontech, from 2 μg of mRNA poly(A)⁺ isolated from various human tissues. Specific antibodies against Erbin were prepared.

Preparation of the Antibodies

The monoclonal antibody anti-Mc 9E10 (Oncogene Research Products, Cambridge, Mass.) was used for the immunoprecipitation and the immunoblot. Monoclonal antibodies of rabbits anti-ERBB2 and anti-EGF-R were obtained by injection of peptides into rabbits. The monoclonal antibody 408 anti-EGF-R was used for the immunoprecipitation. The monoclonal antibody anti-EGF-R (clone LA22) of Upstate Biotechnology (UBI) was used for the immunofluorescence. The polyclonal antibodies anti-SHC and monoclonal antibody 4G10 mAb (anti-PY) were obtained from UBI. Anti-rabbit and anti-mouse goat IgG coupled to a horseradish peroxidase were purchased from Laboratoire Jackson and Dako respectively.

A polyclonal anti-Erbin rabbit antibody was produced by injecting a fusion protein GST-Erbin (914-1371). The anti-Erbin antibody was also purified by affinity with a histidine labeled Erbin (914-1371) binded to beads of agarose and of nickel (Qiagen).

These specific antibodies against Erbin detected a protein of 180 kD in the form of a doublet in the brain, the liver, the kidney, the spleen, the intestines and the skeletal muscles as well as in the epithelial cell lines Caco-2 and MDCK (FIG. 3). The expression of cDNA of Erbin of full length in the COS cells permitted the production of a protein of similar size to the protein detected by the antibody in question in the tissues and the cell extracts.

Cell Culture

The cells COS-1 and MDCK are cultivated in DMEM medium (Dulbecco modified Eagle medium) containing 100 U/ml of penicillin and 100 μg/ml of streptomycin sulphate, complemented with 10% foetal calf serum (FCS). The Caco-2 cells were kept in DMEM medium complemented with 20% of FCS and 1% of non-essential amino acids. All the cellular transfections were carried out using a reactant FIG. 6 according to the recommendations of the manufacturer (Boehringer Mannheim).

The structural homology between Erbin, DENSIN-180, VARTUL, KAA0147 and F26D11.11 led the authors of the invention to consider these proteins as members of a new PDZ family designated LAP for “LRR and PDZ domain proteins”.

### Example 3

Interaction of Erbin with an Activated HER1/2 Receptor

The interaction in vivo between Erbin and the ERBB2 receptor was tested by co-immunoprecipitation in COS cells.

1) HER1/2 was coexpressed transitorily with the polypeptides Erbin, SAP97 or PSD-95 fused to the epitope myc in COS-1 cells. After a lysis, HER1/2 was immunoprecipitated with the antibody anti-EGF-R (clone 408) and the binded proteins were revealed by anti-myc and anti-ERBB2 antibodies respectively. Only Erbin was associated with HER1/2.

2) HER1/2 and EGF-R were coexpressed transitorily with Erbin in the COS-1 cells. After treatment on the EGF the cells were lysed and an aliquot fraction of a protein extract was deposited on SDS-PAGE, subjected to electrophoresis and transferred onto a nitrocellulose membrane. An equal quantity of Erbin and of receptor was found in the lysates. The receptors were immunoprecipitated with an anti-EGF-R antibody, the binded proteins were subjected to a Western Blot and revealed with anti-Erbin and anti-receptor antibodies. Only Her1/2 interacted with Erbin. As control, the protein p52 SHC was immunoprecipitated in an increasing manner with HER1/2 and EGF-R after stimulation by EGF.

3) HER1/2 and EGF-R were expressed transitorily in the COS-1 cells. After 5 minutes of stimulation with medium containing no growth factors or containing 200 ng/ml EGF, the cells were lysed, and “pull-down” GST tests of precipitation of proteins were carried outusing the GST-SHC PTB or GST-ERBIN PDZ domains fixed on beads of agarose. the proteins precipitated were revealed by a Western Blot analysis using an anti-PY antibody (higher screens). After dehybridisation of the antibodies, the membranes were revealed with anti-ERBB2 and anti-EGF-R antibodies respectively (anti-RTK). Whist only the phosphorylated receptors binded to the SHC PTB domain, non phosphorylated HER1/2 interacted with the PDZ domain of Erbin.

4) HER1/2 containing a mutation of the carboxy-terminal valine (mutant VA) or a “kinase-dead” HER1/2, that is to say an HER1/2 deprived of catalytic activity (mutant KA), were expressed in COS-1 cells and the interaction with GST fusion proteins was tested as described previously. The VA mutation in HER1/2 did not affect the phosphorylation of tyrosine residues to the receptor whilst the mutant KA was not phosphorylated on tyrosines. As expected, the mutant HER1/2 KA did not bind to the SHC PTB domain (FIG. 3d). On the other hand, the PDZ domain of Erbin precipitates HER1/2 KA in an effective manner but not via HER1/2 VA. Taken as a whole, these results show that the PDZ domain of Erbin interacts solely with the non-activated HER1/2 receptor.

The authors of the invention wondered whether the proteins behaved in an identical manner in vivo by coexpressing Erbin labeled by myc with HER1/2 in the COS cells. It was found that the pool of HER1/2 associated with Erbin was not phosphorylated at the site of the tyrosine after stimulation with EGF. On the other hand, the SHC proteins interacted with the phosphorylated receptor. Erbin is a substrate for the ERBB2 kinase and no phosphorylation of Erbin was found during the expression of the HER1/2 KA receptor. These data show that Erbin interacts with non-phosphorylated HER1/2 on tyrosine and identify Erbin as a substrate of the ERBB2 kinase. As a SHC PTB binding site was found at the C-terminal end of ERBB2, two residues upstream of the binding site to the PDZ domain (Borg et al, 1978), the recruitment of the SHC proteins at this site, after stimulation with EGF, could prevent the fixation of the PDZ domain of Erbin. The phosphorylation of the tyrosine in itself is not sufficient to alter the interaction of the HER1/2 PDZ domain since the peptide phospshorylated at the level of the tyrosine inhibits this interaction as effectively as a non-phosphorylated peptide. The interaction of the PDZ domain is therefore capable of being inhibited by competition with protein modules of the signalling machinery.

### Example 4

The Basolateral Localisation of ERBB2 in the Epithelial Cells is Dependent upon the Site of Interaction with Erbin

In order to make a closer study of the sub-cellular localisation of ERBB2 and of Erbin, the authors of the invention used the Caco-2 line, a cell line of human colon carcinoma, expressing both proteins.

The fractionation of the Caco-2 cells showed that Erbin was principally present in the membrane fraction whilst the SHC proteins were predominant in the cytolosic fraction. Immunostaining was then carried out on the Caco-2 cells polarised with a purified anti-Erbin antibody. For that, the Caco-2 cells were seeded on glass strips, permeabilised and stained with the indicated primary antibodies, then subjected to staining with the secondary antibodies conjugated to different fluorochromes according to the following protocol.

Immunolocalisation and Labeling of the Cell Surfaces

For the immunostaining procedures, the MDCK cells were cultivated on glass strips for three days after confluence and were labeled as described in Le Bivic et al, 1989, with a monoclonal antibody against EGF-R and a polyclonal antibody against gp114 (apical marker of the MDCK cells) (Le Bivic et al, 1990). The Caco-2 cells were cultivated on glass strips for 10 days after confluence in order to ensure a complete differentiation, then were subjected to the same treatment as the MDCK cells. The antibody against Ag525 (basolateral marker of human intestinal cells) was described previously (Le Bivic et al, 1988). Frozen sections (0.5 to 1 μm) of human colon were obtained as described previously (Le Bivic et al, 1988).

For the biotinylation of the cell surfaces, the cells cultivated on filters for three days after confluence were labeled for one night with “³⁵S-promix ready vue” from Amersham (18.5 MBq/ml) on the basolateral face. The cells were labeled with Sulfo-NHS-LC-biotin (Pierce) with 90 minutes chase in normal medium either on the apical face or on the basolateral face and EGF-R or the chimeras were immunoprecipitated with streptavidine as described in Le Bivic et al, 1989. The specimens were analysed by SDS-PAGE and visualised by fluorography. The autoradiograms were quantified using Intelligent Quantifier from Bioimage (Ann Arbor, Mich.).

Erbin was found on the basolateral face of the plasmic membrane of Caco-2 since the colocalisation was obtained by co-staining with the monoclonal antibody 525, specific marker of the basolateral epithelium.

Moreover, ERBB2 was also basolateral and colocalised with Erbin. The staining of section of human colon with the anti-Erbin antibody confirmed the basolateral localisation of the protein in these tissues.

The authors of the invention then produced MDCK cells expressing EGF-R, HER1/2 and HER1/2.VA in a stable manner. MDCK expressed low levels of endogenous EGF-R and ERBB2 receptors contrary to the cell populations transfected with the constructs, which permitted an expression of EGF-R and HER1/2 (initial and mutant).

Immunofluorescence experiments were carried out with LA22, which is a specific human monoclonal anti-EGF-R antibody. EGF-R and HER1/2 were localised on the basolateral face whilst the mutation of HER1/2 (HER1/2.VA) prevented the basolateral localisation of HER1/2. The biotinylation of the cell surface was carried out on the apical and basolateral faces of the transfectants MDCK and the quantities of receptors were quantified by autoradiography. Whilst EGF-R and HER1/2 were localised at 83% of the basolateral side, the mutant HER1/2.VA was only 53% basolateral. Thus the elimination of the site of interaction of Erbin led to a depeolarisation of ERBB2 in the epithelial cells.

### Example 5

Production of a “Knock-Out” Mouse

The strategy consists of eliminating the first exon from Erbin gene coding for the first 63 amino acids of Erbin and replacing it by a cassette coding for the gene for neomycin resistance. The disappearance of the translation initiation codon and of the following 62 amino acids causes a loss of expression of the functional Erbin protein.

The homologous recombination vector of pPNT3 type substitutes sequences of the Erbin gene (exon 1) by a cassette of gene for neomycin resistance. It comprises:

the gene for resistance to neomycin (neo^{r}) provided with two recognised promoters in eukaryote and prokaryote cells.

the PGK-TK cassette is constituted by the minimal promoter of phosphoglycerate kinase (PGK) and sequences coding the thymidine kinase of the Herpes simplex virus. The PGK promoter is normally activated in the ES cells and permits a negative selection to be made in the course of the selection of the recombinant clones. This cassette is placed in 5′ of the regions of homology of the Erbin. The recombination vector is constructed from 6 kb of genomic sequence of Erbin: 1.5 kb upstream of the first exon (5′ arm) and 4.5 kb (3′ arm) downstream of the first exon. The final recombination vector has the following DNA sequences in series: 5′ Cassette PGK-TK—arm 5′ Erbin—neo^{r} gene—arm 3′ Erbin 3′. After linearisation of the recombination vector, this latter is transferred by electroporation into ES 129 cells (embryonic stem cells) and the transfected cells are selected by addition of geneticine into the culture medium, according to the protocol described in “Gene targeting: a practical approach”, Editions A. L. Joyner 1993 IRL Press. In order to demonstrate the clones of which the genome includes the event of homologous recombination, a counter-selection of ganciclovir is used. The clones are verified according to the protocol proposed in “Gene targeting: a practival approach”, Editions A. L. Joyner 1993 IRL Press, by PCR and Southern Blot, and Fiore et al, Int. J. Dev. Biol. 41: 639-642 (1997).

The selected clones are injected into C57BL/6 mouse blastocytes taken at 3.5 days of gestation. The blastocytes are reimplanted into pseudogestant SWISS mice of 2.5 days. The chimeras are demonstrated by the colour of their fur (black and brown for the chimeras). The chimeric males are crossed with C57BL/6 females and the tails of agouti newborns are taken and their genotype analysed by Southern Blot in order to detect the recombined allele. The crossing of heterozygotic mice amongst themselves makes it possible to obtain homozygotic mutants for the mutation (generation F2) (verification by Southern Blot).

### Example 6

Mutagenesis of the PDZ Domain of Erbin

Punctual mutations effected in the PDZ domain of Erbin have inverse effects on the interaction with ERBB2/HER2.

In order to show the effect of these mutations, the following two techniques in particular can be used: precipitation of ERBB2 by recombinant fusion proteins and double-hybrid system in yeast.

Wild-type or mutated recombinant fusion proteins GST-PDZ ERBIN fixed to solid matrix are incubated with a cellular lysate containing ERBB2/HER2. After two hours, the beads are washed and the complexes resolved by SDS-PAGE. After transfer, ERBB2 is revealed by a specific antibody (Western Blot).

For the double-hybrid system in yeast, the bait is constituted by LEXA-BD fused to the ERBB2 peptide. The prey is constituted by GAL4-AD fused to the PDZ domain of wild-type or mutated Erbin.

These two tests gave identical results: mutants PDZ YD (of histidine 1347 into tyrosine and of glycine (position 1348) into aspartic acid) and NS (of threonine 1316 into asparagine and of arginine (position 1317) into serine) have less affinity for the ERBB2 receptor and mutants HL (of histidine (position 1347) into leucine) and MI (of leucine 1291 into methionine and of phenylalanine (position 1293) into isoleucine) have more affinity for ERBB2.

### BIBLIOGRAPHICAL REFERENCES

Antonarakis S. E., Diagnosis of genetic disorders at the DNA level. N Engl J. Med. 320: 153-163 (1989).

Bellis et al, medecine/sciences, 13:1317-24 (1997).

Benoit et al, PNAS USA, 79, 917-921 (1982).

Borg et al (1996), The Phosphotyrosine Interaction Domains of X11 and FE65 bind to distinct sites on the YENPTY motif of amyloid precursor protein. Molecular and Cellular Biology 16: 6229-6241.

Borg et al (1998), Function of PTB domains. Curr. Top. Microbiol. Immunol. 228:23-38.

Borg et al (1998), Identification of an evolutionarily conserved heterotrimeric protein complex involved in protein targeting. J. Biol. Chem. 273(48): 31633-31636.

Cooper et al, Diagnosis of genetic disease using recombinant DNA, 3^{rd} Edition, Hum Genet., 87: 519-560 (1991).

Fanning, A. S. & Anderson, J. M. PDZ domains and the formation of protein networks at the plasma membrane. Curr. Top. Microbiol. Immunol. 228: 209-233 (1998).

Huber, C. G. et al, Rapid and accurate sizing of DNA fragments by ion-pair chromatography on alkylated nonporous poly(styrenedivinylbenzene) particles. Anal. Chem. 67, 578-585 (1995).

Klapper et al (2000), Biochemical and clinical implications of the ERBB/HER signaling network of growth factor receptors. Adv Cancer Res 77: 25-79.

Köhler and Milstein, Nature, 256, 495-497 (1975).

Kuklin, A. et al, Detection of single-nucleotide polymorphisms with the WAVE™ DNA fragment analysis system. Genetic Testing 1, 201-206 (1997/98).

Le Bivic et al (1988) Characterization of a glycoprotein expressed on the basolateral membrane of human intestinal epithelial cells and cultured colonic cell lines. Eur J Cell Biol 46: 113-120.

Le Bivic et al (1989) Vectorial targeting of apical and basolateral plasma membrane proteins in a human adenocarcinoma epithelial cell line. Proc Natl Acad Sci USA 86: 9313-9317.

Le Bivic et al (1990) Vectorial targeting of an endogenous apical membrane sialoglycoprotein and uvomorulin in MDCK cells. J Cell Biol 110: 1533-1539.

Ross et al (1999), HER-2/neu (c-erb-B2) gene and protein in breast cancer, Am. J. Clin. Pathol. 112: S53-67.

Sambrook et al, Molecular cloning, a laboratory manual, Spring Harbor Laboratory Press, 9.54-62 (1989).

Songyang et al (1997) Recognition of unique carboxyl-terminal motifs by distinct PDZ domains. Science 275: 73-77.

Zhang et al (1996) A yeast three-hybrid-method to clone ternary protein complex components. Analytical Biochemistry, 242, 68-72.

1. Isolated polypeptide, called erbin, comprising a sequence of amino acids chosen from amongst SEQ ID no. 2 or no. 4, the sequence SEQ ID no. 2 deleted from residues 1212 to 1280, or the sequence SEQ ID no. 2 with an insertion between the amino acids 1211 and 1212 of the fragment of sequence SEQ ID no. 7.

2. Isolated nucleic acid, comprising a nucleotide sequence coding for a polypeptide as claimed in claim 1.

3. Nucleic acid as claimed in claim 2 comprising the sequence chosen from amongst SEQ ID no. 1, SEQ ID no. 3, the sequence SEQ ID no. 1 with a deletion of the nucleotides 3957 to 4163, or the sequence SEQ ID no. 1 with an insertion between the nucleotides 3956 and 3957 of the fragment of sequence SEQ ID no. 6.

4. Isolated nucleic acid comprising an isolated nucleic acid sequence chosen from amongst the sequence SEQ ID no. 5, or the sequence comprising the nucleotides 2347 to 2535 on SEQ ID no. 5.

5. Isolated polypeptide, comprising a sequence chosen from amongst:
a) the residues 1 to 501 of SEQ ID no. 2;
the residues 296 to 334 of SEQ ID no. 4;
b) the sequence SEQ ID no. 7;
c) the sequence SEQ ID no. 2 with a mutation of the histidine 1347 into leucine;
the sequence SEQ ID no. 2 with a mutation of the leucine 1291 into methionine and of the phenylalanine 1293 into isoleucine;
the sequence SEQ ID no. 2 with a mutation of the histidine 1347 into tyrosine and of the glycine 1348 into aspartic acid; and
the sequence SEQ ID no. 2 with a mutation of the threonine 1316 into asparagine and of the arginine 1317 into serine.

6. Isolated polypeptide, consisting of a sequence chosen from amongst:
the residues 1279 to 1371 of SEQ ID no. 2;
the residues 502 to 1278 of SEQ ID no. 2
the residues 914 to 1371 of SEQ ID no. 2; and
the residues 1212 to 1280 of SEQ ID no. 2.

7. Isolated nucleic acid comprising a nucleotide sequence coding for the polypeptide of claim 5 or 6.

8. Cloning and/or expression vector containing a nucleic acid as claimed in any one of claims 2, 3, 4 or 7.

9. Host cell transfected by a vector as claimed in claim 8.

10. Use of a nucleic acid as claimed in one of claims 2, 3, 4 or 7 for obtaining probes or triggers having at least 15 nucleotides, which hybridise specifically with the nucleic acid sequence of claims 2, 3, 4 or 7 or its complementary sequence in stringent hybridisation conditions.

11. Nucleic probe of which the sequence is chosen from amongst the sequence ID no. 8 or SEQ ID no. 9.

12. Nucleic trigger of which the sequence is chosen from amongst the sequence SEQ ID no. 10 to SEQ ID no. 20.

13. Process for the production of a recombinant polypeptide, in which a vector containing a nucleic acid as claimed in claim 2, 3, 4 or 7 is transferred into a host cell, which is cultured in conditions permitting the expression of the polypeptide as claimed in claim 1 or 5, or of a polypeptide coded by a nucleic acid sequence such as is defined in claim 4.

14. Process for diagnosis in vitro of a tumour or of a predisposition to develop a tumour, comprising the steps consisting of:
a1) putting a biological specimen containing DNA or RNA into the presence of specific oligonucleotides permitting the amplification of all or part of the erbin gene or of its transcript, comprising a sequence such as is defined in one of claims 2, 3, 4 or 7;
b1) amplifying the said DNA or RNA;
c1) detecting the products of amplification;
d1) comparing the products of amplification which are obtained with those obtained with a control specimen, and in this way detecting a possible anomaly in the erbin gene or in its transcript, indicating a predisposition to develop a tumour, or
a2) putting a biological specimen containing mRNA, obtained by taking a sample of suspect cells from a patient, into the presence of specific oligonucleotides permitting the amplification of all or part of the transcript of the erbin gene, comprising a sequence such as is defined in claim 2, 3, 4 or 6;
b2) amplifying the said transcript;
c2) detecting and quantifying the products of amplification;
a modification in the amount of erbin transcript with reference to the normal control indicating a tumour or a predisposition to develop a tumour.

15. Antibody directed against the polypeptide as defined in claim 1, 5 or 6.

16. Use of at least one antibody as claimed in claim 15 for the detection or the purification of a polypeptide such as is defined in claim 1 or 5 in a biological specimen.

16. Method of diagnosis in vitro of a tumour or of a predisposition to develop a tumour, comprising placing at least one antibody directed against the polypeptide as defined in claim 1, 5 or 6 in contact with a biological specimen obtained from a sample of suspect cells taken from a patient, in conditions permitting the possible formation of specific immunological complexes between the polypeptide as defined in claim 1, 5 or 6 and the said antibody or antibodies and the detection and/or the quantification of the specific immunological complexes which may be formed.

18. Kit comprising:
at least one antibody as claimed in claim 15, possibly fixed on a support;
means for revealing the formation of specific antigen/antibody complexes between the polypeptide of claim 1, 5 or 6 and the said antibody and/or means for quantification of these complexes.

19. Pharmaceutical composition comprising a nucleic acid as claimed in one of claims 2, 3, 4 or 7, an anti-sense nucleic acid of the nucleic acid as claimed in one of claims 2, 3, 4 or 7, or a polypeptide as claimed in claim 1, 5 or 6, in association with a pharmaceutically acceptable vehicle.

20. Use of a nucleic acid as claimed in claim 2, 3, 4 or 7 or of a polypeptide as claimed in claim 1, 5 or 6, for the manufacture of a medication intended for the treatment of tumours.

21. Process for obtaining a non-human transgenic animal in which the gene coding for the polypeptide as defined in claim 1 is invalidated.

22. Non-human transgenic animal capable of being obtained by the process of claim 21.

23. Method of screening molecules inhibiting or activating the bonding between the polypeptide of claim 1 and the erbB2 receptor, comprising:
placing a molecule to be tested in contact with
(i) a first bonding partner which is the erbB2/HER2 receptor or a fragment thereof capable of fixing the polypeptide of claim 1, and
(ii) a second bonding partner which is the polypeptide of claim 1 or a fragment thereof capable of fixing the erbB2/HER2 receptor,
the said first and/or second bonding partner(s) being marked as needed in such a way as to be detected,
evaluating the inhibition or activation of the interaction between the said bonding partners by the molecule to be tested.

24. Process for identification of mutants of the polypeptide of claim 1 or mutants of erbB2/HER2 which increase or decrease the interaction between this protein and the erbB2/HER2 receptor, in which:
either a mutation is effected on the polypeptide of claim 1, 5 or 6 and the effect of this mutation on the interaction of the said polypeptide with the erbB2/HER2 receptor is evaluated;
or a mutation is effected on the erbB2/HER2 receptor and the effect of this mutation on the interaction of the said receptor with the polypeptide of claim 1, 5 or 6 is evaluated.


SEQUENCE LISTING (21 sequences)

SEQ ID NO: 1 - DNA, 6412 bp, Homo sapiens
  CDS (324)..(4436)
aaagatcttttttttttttttttctttttttttcggcggagatcctcgttggggctgggaaactcctgcaaaactcgagaccaggaagccagcccgcaccccaacccccaccaaagccacctactcttcttctgtgggaggccagtccacatccgctctcacccgagagagatattcagctggatccaaagtgactgatgaagggaaggaaatcatgtcaagcgaagccttgaaaaagctgccctgagacggtgtcccgccgaaagaatgttggctcaattaagaaacatcagggagataaattcaacccagtgtgtctaaaaatgactacaaaacgaagtttgtttgtgcggttggtaccatgtcgctgtctacgaggggaagaggagactgtcactactcttgattattctcattgcagcttagaacaagttccgaaagagatttttacttttgaaaaaaccttggaggaactctatttagatgctaatcagattgaagagcttccaaagcaactttttaactgtcagtctttacacaagctgagtttgccagacaatgatttaacaacgttaccagcatccattgcaaaccttattaatctcagggaactggatgtcagcaagaatggaatacaggagtttccagaaaatataaaaaattgtaaagttttgacaattgtggaggccagtgtaaaccctatttccaagctccctgatggattttctcagctgttaaacctaacccagttgtatctgaatgatgcttttcttgagttcttgccagcaaattttggcagattaactaaactccaaatattagagcttagagaaaaccagttaaaaatgttgcctaaaactatgaatagactgacccagctggaaagattggatttgggaagtaacgaattcacggaagtgcctgaagtacttgagcaactaagtggattgaaagagttttggatggatgctaatagactgacttttattccagggtttattggtagtttgaaacagctcacatatttggatgtttctaaaaataatattgaaatggttgaagaaggaatttcaacatgtgaaaaccttcaagacctcctattatcaagcaattcacttcagcagcttcctgagactattggttcgttgaagaatataacaacgcttaaaatagatgaaaaccagttaatgtatctgccagactctataggagggttaatatcagtagaagaactggattgtagtttcaatgaggttgaagctttgccttcatctattgggcagcttactaacttaagaacttttgctgctgatcataattacttacagcagttgcccccagagattggaagctggaaaaatataactgtgctgtttctccattccaataaacttgagacacttccagaggaaatgggtgatatgcaaaaattaaaagtcattaatttaagtgataatagattaaagaatttaccctttagctttacaaagctacagcaattgacagctatgtggctctcagataatcagtccaaacccctgatacctcttcaaaaagaaactgattcagagacccagaaaatggtgcttaccaactacatgttccctcaacagccaaggactgaggatgttatgtttatatcagataatgaaagttttaacccttcattgtgggaggaacagaggaaacagcgggctcaagttgcatttgaatgtgatgaagacaaagatgaaagggaggcacctcccagggagggaaatttaaaaagatatccaacaccatacccagatgagcttaagaatatggtcaaaactgttcaaaccattgtacatagattaaaagatgaagagaccaatgaagactcaggaagagatttgaaaccacatgaagatcaacaagatataaataaagatgtgggtgtgaagacctcagaaagtactactacagtaaaaagcaaagttgatgaaagagaaaaatatatgataggaaactctgtacagaagatcagtgaacctgaagctgagattagtcctgggagtttaccagtgactgcaaatatgaaagcctctgagaacttgaagcatattgttaaccatgatgatgtttttgaggaatctgaagaactttcttctgatgaagagatgaaaatggcggagatgcgaccaccattaattgaaacctctattaaccagccaaaagtcgtagcacttagtaataacaaaaaagatgatacaaaggaaacagattctttatcagatgaagttacacacaatagcaatcagaataacagcaattgttcttctccatctcggatgtctgattcagtttctcttaatactgatagtagtcaagacacctcactctgctctccagtgaaacaaactcatattgatattaattccaaaatcaggcaagaagatgaaaattttaacagccttttacaaaatggagatattttaaacagttcaacagaggaaaagttcaaagctcatgataaaaaagattttaacttacctgaatatgatttgaatgttgaagagcgattagttctaattgagaaaagtgttgactcaacagccacagctgatgacactcacaaattagatcatatcaatatgaatcttaataaacttataactaatgatacatttcaaccagagatcatggaaagatcaaaaacacaggatattgtgcttggaacaagctttttaagcattaattctaaagaggaaactgagcacttggaaaatggaaacaagtatcctaatttggaatccgtaaataaggtaaatggacattctgaggaaacttcccagtctcctaataggactgaaccacatgacagtgattgttctgttgacttaggtatttccaaaagcactgaagatctctcccctcagaaaagtggtccagttggatctgttgtgaaatctcatagcataactaatatggagattggagggctaaaaatctatgatattcttagtgataatggacctcagcagccaagtacaaccgttaaaatcacatctgctgttgatggaaaaaatatagtcaggagcaagtctgccacactgttgtatgatcaaccattgcaggtatttactggttcttcctcatcttctgatttaatatcaggaacaaaggcaattttcaagtttgattcaaatcataatcccgaagagccaaatataataagaggccccacaagtggcccacaatctgcacctcaaatatatggtcctccacagtataatatccaatacagtagcagtgctgcagtcaaagacactttgtggcactccaaacaaaatccccaaatagaccatgccagttttcctcctcagctccttcctagatcagagagcacagaaaatcaaagttatgctaaacattctgccaatatgaatttctctaatcataacaatgttcgagctaatactgcataccatttacatcagagacttggcccagcaagacatggggaaatgtgggccatctcaccaaacgaccgacttattcctgcagtaactcgaagtacaatccagcgacaaagtagtgtgtcctccacagcctctgtaaatcttggtgatccaggctctacaaggcgggctcagattcctgaaggagattatttatcatacagagagttccactcagcgggaagaactcctccaatgatgccaggatcacagagacccctttctgcacgaacatacagcatagatggtccaaatgcatcaagacctcagagtgctcgaccctctattaatgaaataccagagagaactatgtcagttagtgatttcaattattcacggactagtccttcaaaaagaccaaatgcaagggttggttctgagcattctttattagatcctccaggaaaaagtaaagttcctcgtgactggagagaacaagtacttcgacatattgaagccaaaaagttagaaaagatgcctttgagtaatggacagatgggccagcctctcaggcctcaggcaaattatagtcaaatacatcacccccctcaggcatctgtggcaaggcatccctctagagaacaactaattgattacttgatgctgaaagtggcccaccagcctccatatacacagccccattgttctcctagacaaggccatgaactggcaaaacaagagattcgagtgagggttgaaaaggatccagaacttggatttagcatatcaggtggtgtcgggggtagaggaaacccattcagacctgatgatgatggtatatttgtaacaagggtacaacctgaaggaccagcatcaaaattactgcagccaggtgataaaattattcaggctaatggctacagttttataaatattgaacatggacaagcagtgtccttgctaaaaactttccagaatacagttgaactcatcattgtacgagaagtttcctcataagcactgtggacaaaaaaagcggggaagacagcaagatttattggaagatacttacaggggaaattaatattttgactatttttatatataaagaagaactcaaaaaattatgttcaaatttgtacattaatgaaataatggataaaggagactgttgaattcataccatataaaacttgttaggtttttaaacatagcaatcaaggctacaaaaacaaacctgtgttgtttttgtatagattgtaggtttatttttggatttcatatacatgactgaactgtgtgcaaggcaatagttagccttgattttagcccagagacagatggcagagctatctctctcatagcttttatgcccttatttttattcaactggtattaatgtttttctcctgaaactactttttttgatgtgggcaagagatttgaagtgttggcttttgctatgtgcatattgaattgaagagtgagtaggtgaaggtggtgctggtgggttcactttccaaggccagactaaaacagttattttctataaaaatctggaagcaaagaatggggatggggagagctacgtggtagtatgtttttattaggagaataatgcaataaaatatgtaatgtcttttttataaagcaaaaaagacaataattgcatttatgagctcggcaggatctgttcttgtcatagccattgactatacatttgctactggtgattcagtttttaattttttagtcacaggaaatttttaactctactgtagatgcatgtccatgcattttctgtgttatggaaatccactgatttttttttttttttcaaatggtggtacttgcaatctgttttataattagtgctccatttaaatctaatttataatttttattttaagcagcaaatgaaacaaaaatggccagttttaagattgtgttgcctgtaacacaaaatgttacgaaggtttaggaaagcctctttgatttttgtttggccttgcattggcccttggtaaagtaaaaggaaacagtacacttggagctaggaaaccaaagcaagctttgtgaaactggcacagtgatagagaattgctgtggagagttatagagcaaagggatgggtccttgaggcctgccagtgtgtaaaggtgttcaaataaagggctgtttctacaggtaacattaaatgtgaactcaacacttccagagtctttaaagggtttctatgtgtatcagtgtaatagtgttttaccaccaactgcctttctttgttcctagttactgtaacaaatatttgatgatagaggtttattaattttgtttatccagaccattaattttatttgtttttgtctatgtaatcaaataaaatttgagtaacatgtaatggtaaggattaatgcatggttatttggaccagaaaaaagtgccatagaagaccaataactgtttagttgaggctagtctggaacctttcattagagcaatatttggttattgcacttcatttttatttactaagaaatgcaatttgggaatttttaatctgttatgctttgtttatcaaccttgattttaattaagacttttataagactagcttaaaacaccaaccaacattatttttgcaaaagtgagttggactcactttccattcttgctagtcagagtaagtaggcagcacttttaaaaatatgtgaactcaaatattgcacttctttcaagatgttatcaattggttattgtactgtatagttttaataattttgattgaaaccctttaacaactctttgtaaattttaactcattttagttgattttcagtactatttacataggaattgatttttatggatatagtagaagaaatgtgctgtattttgataaaattcacttattgtatgtgtgttgtaatctaaaaaaaaaaagaatgacaaacagcttctttaagacaaaaaaaaaaaaaaaaaaaaaatat

SEQ ID NO: 2 - protein, 1371 aa, Homo sapiens
MTTKRSLFVRLVPCRCLRGEEETVTTLDYSHCSLEQVPKEIFTFEKTLEELYLDANQIEELPKQLFNCQSLHKLSLPDNDLTTLPASIANLINLRELDVSKNGIQEFPENIKNCKVLTIVEASVNPISKLPDGFSQLLNLTQLYLNDAFLEFLPANFGRLTKLQILELRENQLKMLPKTMNRLTQLERLDLGSNEFTEVPEVLEQLSGLKEFWMDANRLTFIPGFIGSLKQLTYLDVSKNNIEMVEEGISTCENLQDLLLSSNSLQQLPETIGSLKNITTLKIDENQLMYLPDSIGGLISVEELDCSFNEVEALPSSIGQLTNLRTFAADHNYLQQLPPEIGSWKNITVLFLHSNKLETLPEEMGDMQKLKVINLSDNRLKNLPFSFTKLQQLTAMWLSDNQSKPLIPLQKETDSETQKMVLTNYMFPQQPRTEDVMFISDNESFNPSLWEEQRKQRAQVAFECDEDKDEREAPPREGNLKRYPTPYPDELKNMVKTVQTIVHRLKDEETNEDSGRDLKPHEDQQDINKDVGVKTSESTTTVKSKVDEREKYMIGNSVQKISEPEAEISPGSLPVTANMKASENLKHIVNHDDVFEESEELSSDEEMKMAEMRPPLIETSINQPKVVALSNNKKDDTKETDSLSDEVTHNSNQNNSNCSSPSRMSDSVSLNTDSSQDTSLCSPVKQTHIDINSKIRQEDENFNSLLQNGDILNSSTEEKFKAHDKKDFNLPEYDLNVEERLVLIEKSVDSTATADDTHKLDHINMNLNKLITNDTFQPEIMERSKTQDIVLGTSFLSINSKEETEHLENGNKYPNLESVNKVNGHSEETSQSPNRTEPHDSDCSVDLGISKSTEDLSPQKSGPVGSVVKSHSITNMEIGGLKIYDILSDNGPQQPSTTVKITSAVDGKNIVRSKSATLLYDQPLQVFTGSSSSSDLISGTKAIFKFDSNHNPEEPNIIRGPTSGPQSAPQIYGPPQYNIQYSSSAAVKDTLWHSKQNPQIDHASFPPQLLPRSESTENQSYAKHSANMNFSNHNNVRANTAYHLHQRLGPARHGEMWAISPNDRLIPAVTRSTIQRQSSVSSTASVNLGDPGSTRRAQIPEGDYLSYREFHSAGRTPPMMPGSQRPLSARTYSIDGPNASRPQSARPSINEIPERTMSVSDFNYSRTSPSKRPNARVGSEHSLLDPPGKSKVPRDWREQVLRHIEAKKLEKMPLSNGQMGQPLRPQANYSQIHHPPQASVARHPSREQLIDYLMLKVAHQPPYTQPHCSPRQGHELAKQEIRVRVEKDPELGFSISGGVGGRGNPFRPDDDGIFVTRVQPEGPASKLLQPGDKIIQANGYSFINIEHGQAVSLLKTFQNTVELIIVREVSS

SEQ ID NO: 3 - DNA, 1647 bp, Mus musculus
  CDS (1)..(1485)
gcgtccggcaagtctgccacactgctgtatgatcagcccctgcaggtgttcactgctgcctcctcatcttctgagttactgtcaggtacaaaggcagttttcaagtttgattcaaatcacaaccctgaagagccagatataataagagctgccacagtatctggcccacagtctacacctcacctgtatggtcccccacaatataatgtccagtacagtggcagtgctacagtgaaagacaccttgtggcaccctaaacagaatccacaaatagatcctgtcagttttcctcctcagcgccttccaagatctgagagtgcagaaaatcacagttatgcaaagcattctgccaacatgaatttctctaaccataacaatgttagagccaatactggataccacttacaacagagacttgccccagccaggcatggagaaatgtgggctatctccccgaatgaccgactagtgccagcagtcactcggactactattcagagacagagtagcgtgtcttcaacggcttctgtaaatctcggtgaccccacaaggcgtactgaaggagattacttatcgtacagagagttacattcaatgggaagaactccagtcatgtcaggatcacagagacctctttctgcacgagcgtacagcatcgatggcccaaatacatccaggcctcagagtgcccgtccctctattaatgaaataccagagagaactatgtcagttagtgatttcaattactcacggactagtccttcaaaaagaccaaatacaagggtcgggtctgaacattctctgttagatcctccaggaaaaagcaaggttcctcatgactggcgggaacaagtactacgacacattgaggccaaaaagttagaaaagcatccgcagacatccagtccaggagagtgttgccaagatgatagattcatgtcagaagagcagaaccacccatcaggtgcacttagtcacagaggtcttccagacagcctgatgaaaatgcctttgagtaatggacaaatgggccagcctctcaggcctcaggcacattatagtcaaacccatcacccccctcaggcatctgtggcaaggcatccctctagagaacaactaattgattatttgatgctgaaagtggcccaccagcctccatatacacatccccattgttctcctagacaaggccatgaactggcaaagcaagagattcgagtaagagtagaaaaggatccagaacttggatttagcatatcaggaggtgtcggcggcagaggaaaccctttcagacctgatgatgatggtatatttgtaacaagggtacaacctgaaggaccagcatcaaaattactacagccaggagataaaattattcaggctaatggctacagttttatcaacattgaacatgggcaagcggtgtccttgttaaaaactttccataatgcagtagatctcatcattgtacgagaagtttcttcataagcactatagacaaaaacaatgggggaagatagcaagatttatggaagacacttgcaagggaaatgactattttgcttatttttatatataaagaagaactcaactgttggtttgaaacttgtacattactgaaataatggaccttgtggttagagggaa

SEQ ID NO: 4 - protein, 495 aa, Mus musculus
ASGKSATLLYDQPLQVFTAASSSSELLSGTKAVFKFDSNHNPEEPDIIRAATVSGPQSTPHLYGPPQYNVQYSGSATVKDTLWHPKQNPQIDPVSFPPQRLPRSESAENHSYAKHSANMNFSNHNNVRANTGYHLQQRLAPARHGEMWAISPNDRLVPAVTRTTIQRQSSVSSTASVNLGDPTRRTEGDYLSYRELHSMGRTPVMSGSQRPLSARAYSIDGPNTSRPQSARPSINEIPERTMSVSDFNYSRTSPSKRPNTRVGSEHSLLDPPGKSKVPHDWREQVLRHIEAKKLEKHPQTSSPGECCQDDRFMSEEQNHPSGALSHRGLPDSLMKMPLSNGQMGQPLRPQAHYSQTHHPPQASVARHPSREQLIDYLMLKVAHQPPYTHPHCSPRQGHELAKQEIRVRVEKDPELGFSISGGVGGRGNPFRPDDDGIFVTRVQPEGPASKLLQPGDKIIQANGYSFINIEHGQAVSLLKTFHNAVDLIIVREVSS

SEQ ID NO: 5 - DNA, 3996 bp, Mus musculus
gcggccgcggtaggatcaggcttgtagtcctaacttccgaggctgaggcaggaggctcacttgcctccaggaggccagccgaggctacatgagaccttgtctcaaacaaacaaacaaacaaacaaacagagtaagtaaagaaaaatctgaatttagtctgacatttgaattactctttttgtagcctttgtccacaaaagttttgagtgtgcatgctatgtggcttcctagaacaaatcacatatttaatactccttttctctattaattgtcctgttataattttgtatccatttgctctgtgaagacaaattattagaaactttcaatttttcaatatcatatccaaaatacagtgtctttcagattacatacattaggtaataacatcccataccattcattatatttctaaatctagaatggtttctgggaaatgcattttttaaaaagccatatcatccaggttcttatacaccgtcactgaacacttatttgttcagacgtgtttgttaatgtatggtatgtgagaatcgtagtttgaatcactgaagtaattctatgcaattgaactttctataatgatgggaatattatgtaatgtttgagccactttggtatccttagtagggaataaggaaatgacattttaaattttatttgatttaaccttaatattaagcatgtatatctactggctaccataccggacatcaggtagcaggttatcttattagtatacaacgataagcttaatccctttttatcagttacattccttattgaatgaattgtttattcctggtggtaaaatgtgcatttttataaacattaagtttttataaacacaaagttttcctctaaaatctttatctagaaataagcatacaaaaaaagagaatagaattcttactctgtgtgtgtgatcggattctaacccaggacaagccatacatggtcatgttgtcattgcctcagatgaaaacatcgggtgtttcacttactagatttttaaaaaatctttaaatagtgtataaatagacctattgatactaggttttgaggatctagttcaaatgtatattatatgctttttcaaaaatctgttttatgagtctgtgtagttctaaaaaagcaactcttaagtaccatgttaaagatctttatttttgactgttgtttgtagttaagaaaatctggttctctatggaagcagtaggatattctctaaaaaattgtaatgaagataaagaaaatatttataaacattatccaaagagctacagcatagtgcctgatgattgagtaaagtcagtgtagaaacagttgtactgggttggctgaagtatcgttaagtgggaagcagagcctttagtgccacattgtatcctgaggtaaggaaaaaccaatgttggttgtatacactttacttaaacattcgcattttgttcataaatgattattcttttaagtaattagagtatttaaagtattgccttgctgattttttgttactttcttatattttatgcctgaggtcttactgatttaactctggcttaatgctgtttaaaagtattgtttctgtacctaaggcaagaattatattcatgtacaaagaattactaaccaattggtgatcattctcattttcttaagtaatttcactgctagtttactcttagcctagttagaatttatacacaacttcttaataaaactggccttgtgaatttttattaattcaaagttggctttctctccctgttaactgagagactgtattttaagtgagtttatgcatatataatcttaaacaacttagaacctataagctagtattagattaaaacaaaattatacttggagtagatgggacttttcatgtaggagaatcataagaaagttaaatgtggttgatctctgatgaacggttgagaagacctgaaaatgtctgcacagtcatttcctattcagaaatgcacagtcaggttcttgactaagagacagaactcttagttggaaattagaggaaaaccttttactgtttttaacagctaagcagacttcacaacgcatggttttataaaaacgtaggagtattttgttttgtgttgtattcttggattgttttgtaaagtaccagagagcctgagtttaatttgaagtccagtagactgtcaactgttgatattaataggctcagttgggtgtatgtggtggtggaggtattgtttgtctgtggcatgtggaattgtgctgtatggacggtcatttgttggaaaagttgttgttatattatatgcattctgatttgtttttatgttcatgctgcagcatttaaaaatgactacaaaacgaagtttgtttgtgcggttggtaccatgtcgctgtctacgaggagaagaggagacagtcactactctagattattctcattgcagcttggaacaagttcccaaagagattttcacctttgaaaaaactttagaggagctctatttagatgctaatcagattgaagagcttccaaaggtatgttaatactatcaagaattatattaggtaatgtctatgcatagcagtttatattcttatattgttgtatatatgtgttcctgtttattggtttagtcttactgtaagcacaattcaaaatgatgtagaactttgttcaagttgtttaagtacttgtgtggataattgaattttatttttacttataacaattaaaatagataatcagtatatatgacttctggaaccattagatttaaccagactaagaaaagtgaactcttactgtaatttctataagcactttatatgcattgctaacattgttacttccattgctacagcttttatggttctgtaacttcttagaggtgggattctgctgttttccattttgtatggaattgatttggagacagtgcatctctgaagtctagtaacaactggtaggggacagattgctgctgtgacgttctttctcagattgccactgtgtttattattgttattttagtaagctcttttttctcttatctgtgtttgttttcctgtgtatgaaaaggttgcagtagctttaaattgtttattacagtctgtatccctctgaatggcagacagggtcttaccttgtagactagactgtcctcaactctcagagatctgcctgcccttgctttccaaggctgtggttgaaggtgtacaccaccttgctctttgaagctgtaaatggagacttgaaattgatggcttaaaaaacaagttagactttggcatggtggtacctaaatgagaaagatttctagttcaaggccagtctgacctacacagtgggttctgtcatggcctggatcatatatataatcagaccttggtttcaaaacctcctgaccatggtcacgaatgaacaatttaatatgcattaccagaattctgaaaaataaactgtaatttggactatgagatttagacgacaaaattatagaatacttagaatttctgatgaaaaagaaatcgtgtaaatgtgcagtgtgtaggataaactaaaaaactgtttatctgaaattcacatttaattgggtgtctcgtaattaatccagcaaccctaactgaaatgtaacaggtaatatatcttgttgcatatagactttatttaatgaaatagttgttttcaatatttatgtttgtgaacattctgaatgacagtattcaaaagcatttgaaagccactgctgtcctaggtaatgaagttttacaaaactttattgagacaggatctcactttgtgatctagacaaacttcaaacttcattgcaatcctcctgcctcagcttcccagatctggggattataggctgagccactatgcctggtttattcttaataatgtggagaagtctagaacttttctatttgtgcacagtaaaggtcttcagtttaacatggatttgtgaatcacacctgatgcctgcgattgtagtg

SEQ ID NO: 6 - DNA, 144 bp, Homo sapiens
agcatgctgtcaaggtcctttaattccaattttactactgtaagcagttttcactgtggcagctctagggatctgcatggcagccagggcagtcttgccttgagtgttgcagacagaagaggttctggtgggcacatttttcga

SEQ ID NO: 7 - protein, 48 aa, Homo sapiens
SMLSRSFNSNFTTVSSFHCGSSRDLHGSQGSLALSVADRRGSGGHIFR

SEQ ID NO: 8 - DNA, 676 bp, Homo sapiens
ctcgagaccaggaagccagcccgcaccccaacccccaccaaagccacctactcttcttctgtgggaggccagtccacatccgctctcacccgagagagatattcagctggatccaaagtgactgatgaagggaaggaaatcatgtcaagcgaagccttgaaaaagctgccctgagacggtgtcccgccgaaagaatgttggctcaattaagaaacatcagggagataaattcaacccagtgtgtctaaaaatgactacaaaacgaagtttgtttgtgcggttggtaccatgtcgctgtctacgaggggaagaggagactgtcactactcttgattattctcattgcagcttagaacaagttccgaaagagatttttacttttgaaaaaaccttggaggaactctatttagatgctaatcagattgaagagcttccaaagcaactttttaactgtcagtctttacacaagctgagtttgccagacaatgatttaacaacgttaccagcatccattgcaaaccttattaatctcagggaactggatgtcagcaagaatggaatacaggagtttccagaaaatataaaaaattgtaaagttttgacaattgtggaggccagtgtaaaccctatttccaagctccctgatggattttctcagctgttaaacctaacccag

SEQ ID NO: 9 - DNA, 366 bp, Homo sapiens
ccattcagacctgatgatgatggtatatttgtaacaagggtacaacctgaaggaccagcatcaaaattactgcagccaggtgataaaattattcaggctaatggctacagttttataaatattgaacatggacaagcagtgtccttgctaaaaactttccagaatacagttgaactcatcattgtacgagaagtttcctcataagcactgtggacaaaaaaagcggggaagacagcaagatttattggaagatacttacaggggaaattaatattttgactatttttatatataaagaagaactcaaaaaattatgttcaaatttgtacattaatgaaataatggataaaggagactgttgaattc

SEQ ID NO: 10 - DNA, 31 bp, Homo sapiens
gggaggccagtccacatccgctctcacccga

SEQ ID NO: 11 - DNA, 32 bp, Homo sapiens
attcagctggatccaaagtgactgatgaaggg

SEQ ID NO: 12 - DNA, 20 bp, Homo sapiens
actacaaaacgaagtttgtg

SEQ ID NO: 13 - DNA, 22 bp, Homo sapiens
gtttcctcagaatgtccattta

SEQ ID NO: 14 - DNA, 29 bp, Homo sapiens
atgtggttcagtcctattaggagactggg

SEQ ID NO: 15 - DNA, 30 bp, Homo sapiens
cagatccaactggaccacttttctgagggg

SEQ ID NO: 16 - DNA, 44 bp, Homo sapiens
cagcagatgtgattttaacggttgtacttggctgctgaggtcca

SEQ ID NO: 17 - DNA, 31 bp, Homo sapiens
gaggccccacaagtggcccacaatctgcacc

SEQ ID NO: 18 - DNA, 33 bp, Homo sapiens
cctcctcagctccttcctagatcagagagcaca

SEQ ID NO: 19 - DNA, 29 bp, Homo sapiens
cttccaataaatcttgctgtcttccccgc

SEQ ID NO: 20 - DNA, 29 bp, Homo sapiens
atagtcaaaatattaatttcccctgtaag

SEQ ID NO: 21 - protein, 9 aa, Artificial sequence
  Description of the artificial sequence C-terminal end of ERBB2/HER2
EYLGLDVPV